(19) United States
(12) Patent Application Publication
(10) Pub. No.: US 2022/0180976 A1
(43) Pub. Date: Jun. 9, 2022

(54) BIOMARKER SELECTION AND MODELING FOR TARGETED MICROBIOMIC TESTING

(71) Applicant: International Business Machines Corporation, Armonk, NY (US)

(72) Inventors: ANNA PAOLA CARRIERI, Manchester (GB); Niina Haiminen, Valhalla, NY (US); Laxmi Parida, Mohegan Lake, NY (US)

(21) Appl. No.: 17/115,638

(22) Filed: Dec. 8, 2020

(51) Int. Cl. G16B 50/30; G16B 5/00; C12Q 1/6888

(52) U.S. Cl. CPC G16B 50/30; C12Q 1/6888; G16B 5/00

(57) ABSTRACT

A system, method, and computer program product that includes a computer readable storage medium with program instructions, executable by a processer, to cause a device to perform the method. The method includes receiving a set of biomarkers associated with a known phenotype, generating at least one ranking for each biomarker based on a feature selection method, selecting a set of potential key biomarkers from the set of biomarkers based on the ranking, and selecting a set of key biomarkers from the potential key biomarkers. The method also includes building a model for phenotype prediction based on the set of key biomarkers.

### BACKGROUND

The present disclosure relates to machine-learning models for microbiomic testing and, more specifically, to selection of key microbiomic biomarkers for phenotype prediction.

Biomarkers are characteristics that can be evaluated and measured as indicators of physiological states, such as pathogenic processes, normal biological processing, responses to therapeutic treatments, etc. For example, rheumatoid factors detected in a blood sample can be used to diagnose rheumatoid arthritis. Genomic biomarker evaluation can include quantitative analysis of gene expression, detection of gene mutations and polymorphisms, etc. Biomarkers can also be found in microbiota, which are groups of microorganisms (e.g., eukaryotes, archaea, bacteria, fungi, and viruses) that exist within given habitats/hosts (e.g., multicellular organisms, parts of organisms, natural environments, objects, etc.). The aggregated genomes of a microbiota's member organisms are referred to as a microbiome. As is the case with a single organism's genome, there are differences between the microbiomes of different hosts. The microbiome of a host can also vary according to environmental changes, diet, disease state, etc.

### SUMMARY

Various embodiments are directed to a system that includes at least one processing component, at least one memory component, training data, and a training module. The training data includes a set of biomarkers associated with a known phenotype. The training module includes a biomarker and a model generator. The biomarker selector is configured to receive the set of biomarkers, generate at least one ranking for each of the biomarkers based on a feature selection method, and select a set of potential key biomarkers based on the at least one ranking. The at least one ranking can include a correlation value. The biomarker selector selects a set of key biomarkers from the set of potential key biomarkers. In some embodiments, the key biomarkers are selected via graph-based tuning techniques. The biomarker selector can group the set of potential key biomarkers into clusters, select a potential key biomarker from at least one of the clusters, and add this potential key biomarker to the set of key biomarkers. The model generator is configured to build a model for the known phenotype based on the set of key biomarkers. In some embodiments, the model generator is configured to apply the model to a subset of the training data, predict a phenotype associated with the subset, evaluate performance of the model based on the prediction, and determine that the performance is below a threshold performance value. Based on this determining, the biomarker can select additional key biomarkers. The system can also include a testing module configured to receive a microbiota sample. The testing module can identify, via targeted testing, the set of key biomarkers in the microbiota sample and predict, based on the identification, a phenotype associated with the microbiota sample.

Further embodiments are directed to a method, which includes receiving a set of biomarkers associated with a known phenotype, generating at least one ranking for each biomarker based on a feature selection method, selecting a set of potential key biomarkers from the set of biomarkers based on the ranking, and selecting a set of key biomarkers from the potential key biomarkers. In some embodiments, at least one ranking is a correlation value. Selecting the key biomarkers can include graph-based tuning techniques. The set of potential key biomarkers can be sorted into clusters, a potential key biomarker can be selected from at least one of the clusters, and this potential key biomarker can be added to the set of key biomarkers. The method also includes building a model for phenotype prediction based on the set of key biomarkers. The method can include receiving a microbiota sample, identifying the key biomarkers in the microbiota sample via targeted testing, and predicting a phenotype associated with the microbiota sample based on the identification. In some embodiments, the method includes applying the model to a subset of the training data, predicting a phenotype associated with the subset of training data, evaluating performance of the model, and determining that the performance is below a threshold value. In response to this determining, additional key biomarkers can be selected.

Additional embodiments are directed to a computer program product, which includes a computer readable storage medium having program instructions embodied therewith, the program instructions executable by a processor to cause a device to perform a method. The method includes receiving a set of biomarkers associated with a known phenotype, generating at least one ranking for each biomarker based on a feature selection method, selecting a set of potential key biomarkers from the set of biomarkers based on the ranking, and selecting a set of key biomarkers from the potential key biomarkers. Selecting the key biomarkers can include graph-based tuning techniques. The set of potential key biomarkers can be sorted into clusters, a potential key biomarker can be selected from at least one of the clusters, and this potential key biomarker can be added to the set of key biomarkers. The method also includes building a model for phenotype prediction based on the set of key biomarkers. The method can include receiving a microbiota sample, identifying the key biomarkers in the microbiota sample via targeted testing, and predicting a phenotype associated with the microbiota sample based on the identification. In some embodiments, the method includes applying the model to a subset of the training data, predicting a phenotype associated with the subset of training data, evaluating performance of the model, and determining that the performance is below a threshold value. In response to this determining, additional key biomarkers can be selected.

### BRIEF DESCRIPTION OF THE DRAWINGS

FIG. 1 is a block diagram illustrating a microbiomic biomarker evaluation environment, according to some embodiments of the present disclosure.

FIG. 2 is a schematic diagram illustrating graph-based pruning of potential biomarkers, according to some embodiments of the present disclosure.

FIG. 3 is a flow diagram illustrating a process of key biomarker selection and phenotype prediction, according to some embodiments of the present disclosure.

FIG. 4 is a block diagram illustrating a computer system, according to some embodiments of the present disclosure.

FIG. 5 is a block diagram illustrating a cloud computing environment, according to some embodiments of the present disclosure.

FIG. 6 is a block diagram illustrating a set of functional abstraction model layers provided by the cloud computing environment, according to some embodiments of the present disclosure.

### DETAILED DESCRIPTION

Biomarkers are characteristics that can be evaluated and measured as indicators of physiological states, such as pathogenic processes, normal biological processing, responses to therapeutic treatments, etc. Biomarkers can be used in the detection, diagnosis, prognosis, and/or prediction of various diseases. For example, rheumatoid factors detected in a blood sample can be used to diagnose rheumatoid arthritis. Molecular biomarkers such as glucose and hemoglobin A1c can be used to diagnose and monitor the progression of diabetes. Additional biomarkers can be detected/measured using techniques such as medical imaging (e.g., computed tomography, magnetic resonance imaging, etc.) and genomic testing. Genomic biomarker evaluation can include quantitative analysis of gene expression, detection of gene mutations and polymorphisms, etc. For example, there are gene mutations that can indicate a greater likelihood of developing a related disease. Additionally, personalized therapeutic treatments can be based on features of an individual's genomic information.

Biomarkers can also be found in microbiota, which are groups of microorganisms (e.g., eukaryotes, archaea, bacteria, fungi, and viruses) that exist within given habitats (hosts), such as an organism (e.g., a human, a fish, a plant, etc.), part of an organism (e.g., an intestine, skin, leaf, etc.), a natural environment (e.g., water from a lake, soil from an agricultural growing region, etc.), or any other object or region that can support microbial life (e.g., a cosmetic product, a countertop, an article of clothing, air from a duct system, a beverage or food item, etc.). The aggregated genomes (metagenome) of a microbiota's member organisms are referred to as a microbiome. As is the case with a single organism's genome, there are differences between the microbiomes of different hosts. The microbiome of a host can also vary according to environmental changes, diet, disease state, etc.

Therefore, microbiomic information can provide biomarkers for various phenotypes. A phenotype is an observable characteristic or trait of a microbiota or host organism. Models for predicting phenotypes based on associated microbiomic signatures can be built. These models have the potential to identify biological states with great specificity and accuracy. However, there are challenges to evaluating microbiomic data that differentiate it from single-organism genomic testing. For example, microbiomic data often includes false positives, false negatives, variability due to incomplete databases, sampling variation, contamination of sequencing reagents, etc. Further, while a genome is mostly static, the composition of a microbiome can vary dynamically.

Present approaches to microbiomic testing include techniques for measuring the presence and abundance of particular groups of members in a microbiota sample based on microbiomic sequencing. One example of these is shotgun sequencing, an untargeted approach wherein fragments of DNA from all microorganisms in the sample are randomly selected. The random fragments are then reassembled by a computer program for finding overlapping fragment ends. Another approach to testing includes targeted sequencing, wherein a defined region is sequenced for all microbiota sample members having the region in their genomes. For example, 16S ribosomal ribonucleic acid (rRNA) gene sequencing can be used to identify many species of bacteria. This is because bacterial 16S rRNA genes are highly conserved and have variable regions with species-specific signatures. However, the volume and variability of genetic material found in a microbiome sample limit the efficacy and practicality of both untargeted and targeted approaches.

Disclosed herein are techniques for efficiently identifying microbiomic biomarkers associated with phenotypes, and building machine-learning models for phenotype prediction based on targeted microbiomic testing. The biomarkers are selected from a set of training data, which includes biological samples with sequenced microbiomes and associated phenotype values. A limited number of biomarkers from the training data are selected via feature selection followed by graph-based pruning in order to generate a set of key biomarkers for the corresponding phenotype. For example, the key biomarkers can be selected based on features such as abundance, prevalence, correlation, variance, etc. A predictive model is trained to associate sets of key biomarkers with phenotypes. A new microbiota sample can then be tested for various phenotypes by searching for key biomarkers, rather than by evaluating all microbiomic data in the sample.

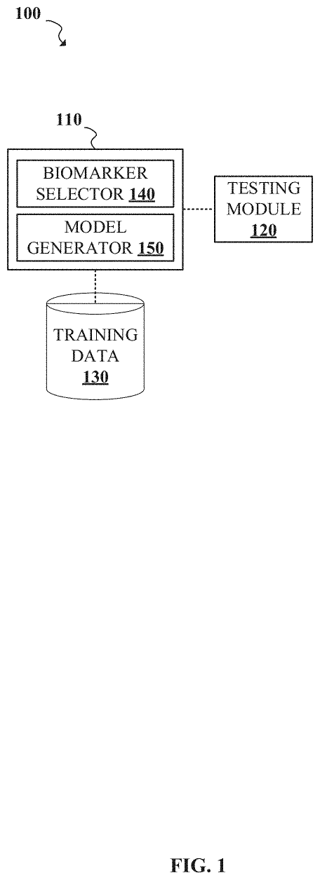

FIG. 1 is a block diagram illustrating a microbiomic biomarker evaluation environment 100, according to some embodiments of the present disclosure. The microbiomic biomarker evaluation environment 100 includes a training module 110, a testing module 120, and a set of training data 130. The training module 110 includes a biomarker selector 140 and a model generator 150

The training data 130 includes labeled microbiomic data. The training data 130 can be obtained from existing databases, such as publicly available microbiome databases. The training data 130 can also be from one or more samples of microbiota obtained from host(s) by a user in order to build a predictive model for a phenotype expressed by the host(s). For example, microbiota from skin swabs or saliva samples can be obtained by users who collect (e.g., via shotgun sequencing, targeted sequencing, etc.) microbiomic data from the samples. The microbiomic data can be labeled with the known phenotype, and added to the training data 130.

The biomarker selector 140 receives an initial set of biomarkers |X| for a known phenotype from the training data 130. The set of biomarkers |X| can include all available microbiomic data associated with the phenotype. The biomarker selector 140 then uses feature selection methods and graph-based pruning to identify a smaller set of biomarkers X (“key biomarkers”) that can be used to build a model of the phenotype. For example, feature selection methods M₁, . . . , M_{K} can be used to obtain a set of rankings F₁, . . . , F_{K} of the biomarkers in the set |X|. For example, the biomarker selector 140 can use methods based on correlation (e.g., Pearson and/or Spearman correlation), feature importance (e.g., Random Forest), permutation importance (ELI5 method), statistics tests (e.g., Chi-Square), wrapper methods (e.g., Recursive Feature Elimination), etc. to assign rankings to each biomarker.

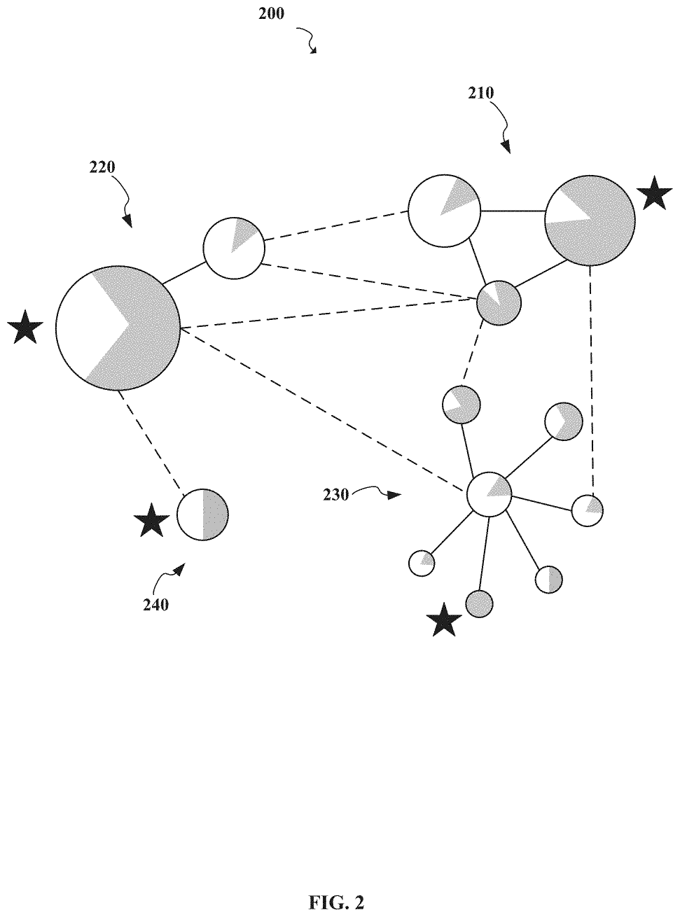

The biomarker selector 140 selects a set of potential key biomarkers F based on the combined rankings of each biomarker in the original set |X|. For example, biomarkers having combined rankings above ranking threshold(s) and/or a given number of biomarkers having the highest combined rankings can be selected. The biomarker selector 140 then applies graph-based pruning techniques to the set of potential key biomarkers F in order to select the set of key biomarkers X The graph-based pruning reduces redundancy and identifies the most robust biomarkers for modeling the phenotype. Sets of key biomarkers are also referred to herein as signature biomarkers for given phenotypes. Selection of key biomarkers via graph-based pruning is discussed in greater detail with respect to FIG. 2.

The model generator 150 builds a model for predicting the phenotype based on the set of key biomarkers X identified by the biomarker selector 140. For example, a phenotype can be associated with key biomarkers that include characteristic species-level relative abundances, presence/absence of species- and/or strain-specific markers, etc. A variety of machine learning techniques can be used to build the predictive model. Examples of these techniques can include Random Forests (RF), Support Vector Machine (SVM), Relevance Vector Machines (RVM), Neural Networks (NN), LightGBM, XGBoost, Lasso, etc.

The model generator 150 can also evaluate the performance of predictive models. For example, a model can be applied to a subset of training data 130 in order to determine the accuracy of the model's phenotype prediction (e.g., based on an F₁-score for classification task or Mean Absolute Error (MAE) for regression task). If the performance of a model is below a given performance threshold (e.g., an F₁-score below 0.7), the biomarker selector 140 can select additional key biomarkers on which to train the model. In some embodiments, these can be selected based on rankings determined for the original set of biomarkers, followed by graph-based pruning of the resulting larger set of potential biomarkers. In other embodiments, additional key biomarkers are selected from the existing set of potential biomarkers F. The model generator 150 can then test the performance of the updated model. If the performance of the updated model is below the performance threshold, the biomarker selector 140 can add more key biomarkers. This can be repeated until the model's phenotype prediction is above the performance threshold or until the number of key biomarkers has reached a practical or financial limit.

The testing module 120 uses models built by the model generator 150 to evaluate data from samples of microbiota. A microbiota sample (e.g., a saliva sample) is obtained from a host. The testing module 120 extracts microbiomic data from the sample in order to test for a phenotype. The testing module 120 can use a targeted approach to data extraction based on the key biomarkers of the phenotype of interest. For example, a sample can be tested for a given host phenotype, Phenotype A. The signature biomarkers of Phenotype A can be relative abundances of two bacterial species, B1 and B2, and the presence of a viral species, V1.

The testing module 120 can therefore determine the relative abundances of B1 and B2 and whether V1 is present. In some embodiments, the presence of V1 can be defined as any detectable quantity of V1. The presence of V1 can also be defined as a quantity of V1 above a threshold quantity. The biomarker measured by relative abundances of B1 and B2 can be defined as, e.g., the quantity of B1 being greater than that of B2. In other embodiments, the biomarker can be defined as a predetermined value or range, such as where the quantity of B1 is at least twice the quantity of B2. Based on these relative abundances and the predictive model for Phenotype A, the testing module 120 can determine the likelihood of the phenotype being expressed by the host. In other embodiments, the testing module 120 can determine likelihoods for multiple phenotypes. For example, the model generator 150 can generate models for more than one phenotype labeled in the training data 130. The testing module 120 can then predict phenotypes based on targeted testing of key biomarkers for multiple phenotypes.

The phenotype predictions can be reported via a user interface (not shown). For example, the most likely phenotype(s) can be reported. In some embodiments, the prediction can be a binary result (e.g., presence or absence of a particular virus). Additionally, the user interface can indicate that results are inconclusive when a prediction cannot be made with sufficient confidence (e.g., when a sample does not include detectable quantities of genetic material). Additionally, the user interface can display values determined for each tested biomarker (e.g., microbiota species, relative abundances, presence/absence, etc.), confidence values, etc. In some embodiments, the microbiome-based phenotype predictions can be reported with other biomarkers (e.g., molecular, genetic, image-based, etc.) and/or information such as patient identity, medical history, reported symptoms, etc. Additional models that combine microbiome biomarker data/phenotype predictions with other biomarkers and/or patient information may also be used to provide phenotype predictions.

FIG. 2 is a schematic diagram 200 illustrating graph-based pruning of potential biomarkers, according to some embodiments of the present disclosure. To illustrate diagram 200, but not to limit embodiments, FIG. 2 is described within the context of the microbiomic biomarker evaluation environment 100 of FIG. 1. Where elements referred to in FIG. 2 are identical to elements shown in FIG. 1, the same reference numbers are used in both Figures.

A set of potential key biomarkers F selected by the biomarker selector 140 (FIG. 1) is represented by circles and edges in FIG. 2. The circles represent microbe species, where the size of each circle represents the species abundance a, and the gray portion of each circle represents the prevalence b of the species. Prevalence can be based on the fraction of samples that contain the species of microbe. The edges represent correlations between microbe species. Solid edges can represent correlations above 0.7, and dashed edges can represent correlations between 0.5 and 0.7. The microbe species are clustered based on correlation so that there are four clusters 210, 220, 230, and 240 of microbe species with correlations above a threshold correlation (0.7). The biomarker selector 140 selects a key biomarker species from each cluster based on abundance a and prevalence b. For example, the microbe species can be ranked by decreasing values of a function of a and b, such as the product of a and b (a*b). In FIG. 2., the species in each cluster having the highest values of a*b are indicated by stars next to their circles. In some embodiments, more than one species can be selected from the same cluster. For example, the selected species can be those with values of a*b above a threshold value.

In other embodiments, the graph-based pruning can be applied to the original set of biomarkers |X| from the training data 130. For example, the biomarkers can be clustered using correlation as a similarity measure (e.g., by a k-means algorithm). Graph-based pruning of the original set of biomarkers |X| may be carried out when fewer than a threshold number of clusters are obtained by pruning the set of potential key biomarkers F. In other embodiments, there can be a greater number of clusters than the desired number of microbes for the set of key biomarkers X (e.g., a number greater than a practical limit for testing). In these instances, the clusters can be ranked by decreasing value based on a function of cluster size, abundance, and prevalence (e.g., max(a*b)*clusterSize), and at most one species per cluster can be selected by the biomarker selector. In some embodiments, no species are selected from clusters with rankings below a threshold ranking.

In instances where the phenotype associated with the set of biomarkers |X| is a categorical phenotype with at least one phenotype value k, abundance a and prevalence b can be defined per phenotype value k by considering only data from samples with the given phenotype. The product can be calculated per microbe species and per phenotype value. The microbe species can then be ranked by decreasing value of a function of their abundance a and prevalence b per phenotype value k (e.g., a_k*b_k). For example, there can be a binary phenotype with values k={1,2}. In this example, microbe species can be ranked by decreasing value of a*b, where a*b=max{a_1*b_1, a_2*b_2}. The biomarker selector 140 can select key biomarkers from the highest ranking (e.g., above a threshold value of a*b) microbe species.

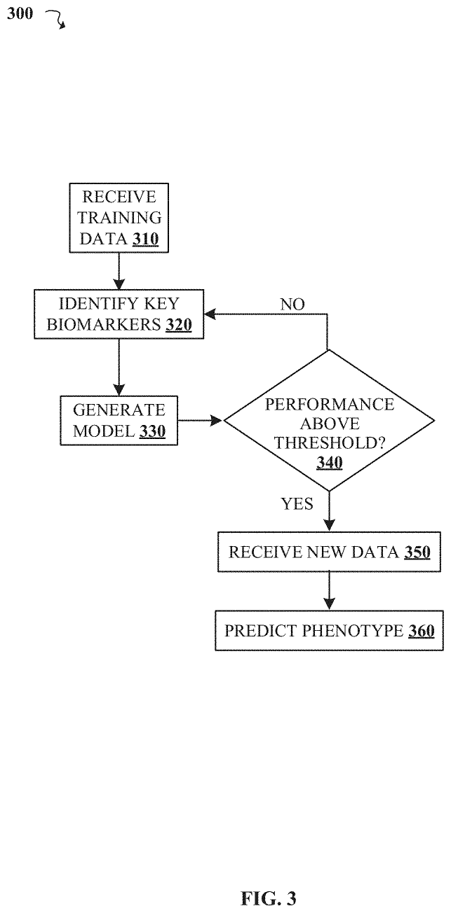

FIG. 3 is a flow diagram illustrating a process 300 of key biomarker selection and phenotype prediction, according to some embodiments of the present disclosure. To illustrate process 300, but not to limit embodiments, FIG. 3 is described within the context of the microbiomic biomarker evaluation environment 100 of FIG. 1. Where elements referred to in FIG. 3 are identical to elements shown in FIG. 1, the same reference numbers are used in each Figure.

The training module 110 receives training data 130. This is illustrated at operation 310. The received training data 130 includes a set of biomarkers for a known phenotype. In some embodiments, there can be training data 130 with biomarkers for more than one phenotype. The training data 130 can be microbiomic data from microbiota samples associated with known phenotypes, which can be gathered and sequenced by one or more users. In some embodiments, the training data 130 is from a public database of known microbiome sequences. The microbiomic training data 130 includes genomic sequences of members of microbiota samples from at least one host or habitat (e.g., an organism, part of an organism, a natural environment, etc.). A microbiota can include various species of microbes, such as bacteria, fungi, archaea, viruses, etc.

Key biomarkers for given phenotype(s) are identified. This is illustrated at operation 320. The biomarker selector 140 can identify a biomarker signature for a phenotype by selecting key biomarkers from an initial set of associated biomarkers from the training data 130. The biomarker selector 140 can use feature selection methods to obtain rankings for biomarkers in this set. For example, features such as microbe species can be ranked based on relative abundance (e.g., of microbe species, strains, genera, etc.), prevalence (e.g., the number of microbiota samples containing a given microbe species), correlation (e.g., between microbe species abundance and phenotype), etc. A set of potential key biomarkers can be selected based on these combined rankings. Graph-based pruning techniques are then used to select a set of key biomarkers for the phenotype from the potential key biomarkers. This is discussed in greater detail with respect to FIGS. 1 and 2.

A predictive model for the phenotype is generated based on the selected key biomarkers. This is illustrated at operation 330. The model generator 150 can use any appropriate machine learning techniques to build the model. Examples of machine learning techniques are discussed in greater detail with respect to FIG. 1. The model generator 150 also tests the performance of the models. This is illustrated at operation 340. The testing module 120 can apply the model to a subset of the training data 130 in order to make a phenotype prediction. The model generator 150 then determines whether the performance of the model is above a threshold value (e.g., an F₁-score or another accuracy measurement value, etc.). If the performance of the model is below the threshold value, additional biomarkers can be added to the set of key biomarkers for the phenotype. Selection of additional biomarkers is discussed in greater detail with respect to FIG. 1. The updated model can again be tested at operation 350, and these steps can be repeated until the performance of the model is above the threshold. However, in some embodiments, the number of key biomarkers can reach limit above which testing would be impractical. In instances where this limit is reached before sufficient accuracy is achieved, process 300 can end at operation 340.

However, if the performance of the model on the training data 130 is above the threshold, new microbiomic data can be received. This is illustrated at operation 350. The testing module 120 can obtain the new data by targeted testing of key biomarkers in a microbiome sample. Based on the microbiomic data, the testing module 120 can use the model generated at operation 330 to make a phenotype prediction for the sample. This is illustrated at operation 360. The phenotype prediction can include likelihoods of one or more phenotypes being expressed by the host or microbiota of the microbiome sample. For example, relative abundances of two microbe species, Species A and B, may be key biomarkers for a phenotype such as a disease state. If there is a greater abundance of Species A than Species B in the sample, the testing module 120 may predict that the disease state phenotype will be expressed by the host. However, if there is a greater abundance of Species B relative to A, the testing module 120 may predict that the phenotype will not be expressed. In some embodiments, the testing module 120 can add the biomarkers from the new microbiomic data to the training data 130. The model generator 150 can optionally update the model with the key biomarkers from the sample testing.

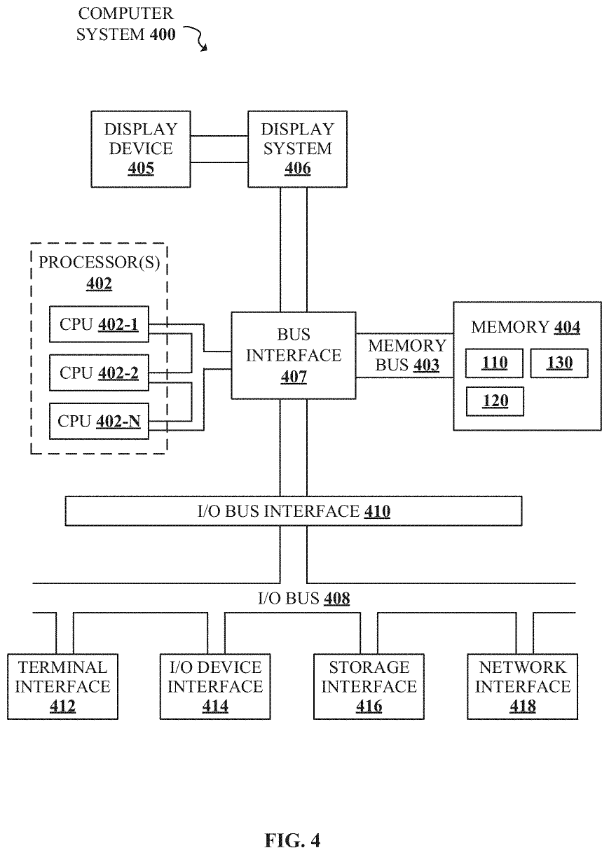

FIG. 4 is a block diagram illustrating an exemplary computer system 400 that can be used in implementing one or more of the methods, tools, components, and any related functions described herein (e.g., using one or more processor circuits or computer processors of the computer). In some embodiments, the major components of the computer system 400 comprise one or more processors 402, a memory subsystem 404, a terminal interface 412, a storage interface 416, an input/output device interface 414, and a network interface 418, all of which can be communicatively coupled, directly or indirectly, for inter-component communication via a memory bus 403, an input/output bus 408, bus interface unit 407, and an input/output bus interface unit 410.

The computer system 400 contains one or more general-purpose programmable central processing units (CPUs) 402-1, 402-2, and 402-N, herein collectively referred to as the CPU 402. In some embodiments, the computer system 400 contains multiple processors typical of a relatively large system; however, in other embodiments the computer system 400 can alternatively be a single CPU system. Each CPU 402 may execute instructions stored in the memory subsystem 404 and can include one or more levels of on-board cache.

The memory 404 can include a random-access semiconductor memory, storage device, or storage medium (either volatile or non-volatile) for storing or encoding data and programs. In some embodiments, the memory 404 represents the entire virtual memory of the computer system 400, and may also include the virtual memory of other computer systems coupled to the computer system 400 or connected via a network. The memory 404 is conceptually a single monolithic entity, but in other embodiments the memory 404 is a more complex arrangement, such as a hierarchy of caches and other memory devices. For example, memory may exist in multiple levels of caches, and these caches may be further divided by function, so that one cache holds instructions while another holds non-instruction data, which is used by the processor or processors. Memory can be further distributed and associated with different CPUs or sets of CPUs, as is known in any of various so-called non-uniform memory access (NUMA) computer architectures.

The training module 110, testing module 120, and training data 130 (FIG. 1) are illustrated as being included within the memory 404 in the computer system 400. However, in other embodiments, some or all of these components may be on different computer systems and may be accessed remotely, e.g., via a network. The computer system 400 may use virtual addressing mechanisms that allow the programs of the computer system 400 to behave as if they only have access to a large, single storage entity instead of access to multiple, smaller storage entities. Thus, though the training module 110, testing module 120, and training data 130 are illustrated as being included within the memory 404, components of the memory 404 are not necessarily all completely contained in the same storage device at the same time. Further, although these components are illustrated as being separate entities, in other embodiments some of these components, portions of some of these components, or all of these components may be packaged together.

In an embodiment, the training module 110, testing module 120, and training data 130 include instructions that execute on the processor 402 or instructions that are interpreted by instructions that execute on the processor 402 to carry out the functions as further described in this disclosure. In another embodiment, the training module 110, testing module 120, and training data 130 are implemented in hardware via semiconductor devices, chips, logical gates, circuits, circuit cards, and/or other physical hardware devices in lieu of, or in addition to, a processor-based system. In another embodiment, the training module 110, testing module 120, and training data 130 include data in addition to instructions.

Although the memory bus 403 is shown in FIG. 4 as a single bus structure providing a direct communication path among the CPUs 402, the memory subsystem 404, the display system 406, the bus interface 407, and the input/output bus interface 410, the memory bus 403 can, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the input/output bus interface 410 and the input/output bus 408 are shown as single respective units, the computer system 400 may, in some embodiments, contain multiple input/output bus interface units 410, multiple input/output buses 408, or both. Further, while multiple input/output interface units are shown, which separate the input/output bus 408 from various communications paths running to the various input/output devices, in other embodiments some or all of the input/output devices may be connected directly to one or more system input/output buses.

The computer system 400 may include a bus interface unit 407 to handle communications among the processor 402, the memory 404, a display system 406, and the input/output bus interface unit 410. The input/output bus interface unit 410 may be coupled with the input/output bus 408 for transferring data to and from the various input/output units. The input/output bus interface unit 410 communicates with multiple input/output interface units 412, 414, 416, and 418, which are also known as input/output processors (IOPs) or input/output adapters (IOAs), through the input/output bus 408. The display system 406 may include a display controller. The display controller may provide visual, audio, or both types of data to a display device 405. The display system 406 may be coupled with a display device 405, such as a standalone display screen, computer monitor, television, or a tablet or handheld device display. In alternate embodiments, one or more of the functions provided by the display system 406 may be on board a processor 402 integrated circuit. In addition, one or more of the functions provided by the bus interface unit 407 may be on board a processor 402 integrated circuit.

In some embodiments, the computer system 400 is a multi-user mainframe computer system, a single-user system, or a server computer or similar device that has little or no direct user interface, but receives requests from other computer systems (clients). Further, in some embodiments, the computer system 400 is implemented as a desktop computer, portable computer, laptop or notebook computer, tablet computer, pocket computer, telephone, smart phone, network switches or routers, or any other appropriate type of electronic device.

It is noted that FIG. 4 is intended to depict the representative major components of an exemplary computer system 400. In some embodiments, however, individual components may have greater or lesser complexity than as represented in FIG. 4, Components other than or in addition to those shown in FIG. 4 may be present, and the number, type, and configuration of such components may vary.

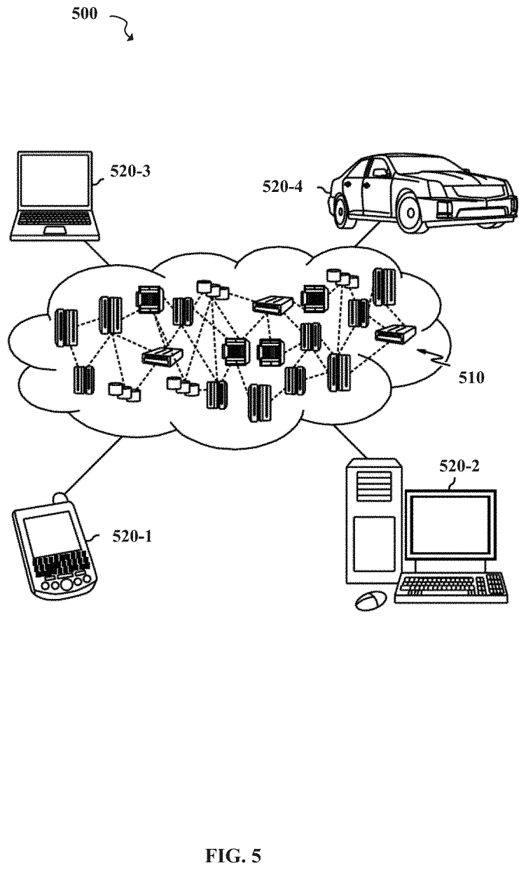

In some embodiments, the data storage and retrieval processes described herein could be implemented in a cloud computing environment, which is described below with respect to FIGS. 4 and 5. It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher-level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

FIG. 5 is a block diagram illustrating a cloud computing environment 500, according to some embodiments of the present disclosure. As shown, cloud computing environment 500 includes one or more cloud computing nodes 510 with which local computing devices used by cloud consumers, such as, for example, personal digital assistant (PDA) or cellular telephone 520-1, desktop computer 520-2, laptop computer 520-3, and/or automobile computer system 520-4 may communicate. Nodes 510 may communicate with one another. They may be grouped (not shown) physically or virtually, in one or more networks, such as Private, Community, Public, or Hybrid clouds as described hereinabove, or a combination thereof. This allows cloud computing environment 500 to offer infrastructure, platforms and/or software as services for which a cloud consumer does not need to maintain resources on a local computing device. It is understood that the types of computing devices 520-1-520-4 shown in FIG. 5 are intended to be illustrative only and that computing nodes 510 and cloud computing environment 500 can communicate with any type of computerized device over any type of network and/or network addressable connection (e.g., using a web browser).

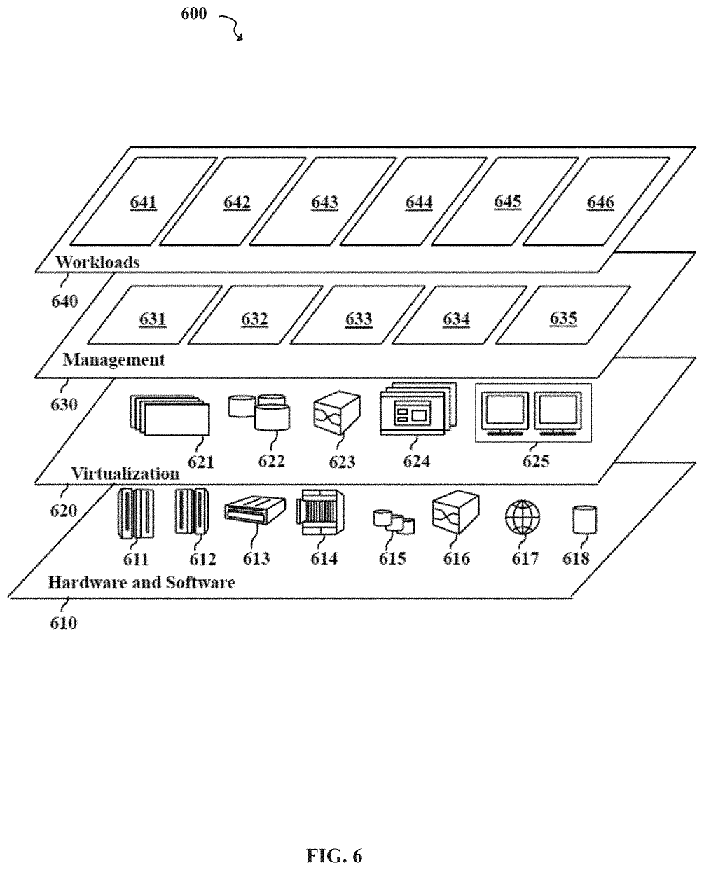

FIG. 6 is a block diagram illustrating a set of functional abstraction model layers 600 provided by the cloud computing environment 500, according to some embodiments of the present disclosure. It should be understood in advance that the components, layers, and functions shown in FIG. 6 are intended to be illustrative only and embodiments of the invention are not limited thereto. As depicted, the following layers and corresponding functions are provided:

Hardware and software layer 610 includes hardware and software components. Examples of hardware components include: mainframes 611; RISC (Reduced Instruction Set Computer) architecture-based servers 612; servers 613; blade servers 614; storage devices 615; and networks and networking components 616. In some embodiments, software components include network application server software 617 and database software 618.

Virtualization layer 620 provides an abstraction layer from which the following examples of virtual entities may be provided: virtual servers 621; virtual storage 622; virtual networks 623, including virtual private networks; virtual applications and operating systems 624; and virtual clients 625.

In one example, management layer 630 provides the functions described below. Resource provisioning 631 provides dynamic procurement of computing resources and other resources that are utilized to perform tasks within the cloud computing environment. Metering and Pricing 632 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 633 provides access to the cloud computing environment for consumers and system administrators. Service level management 634 provides cloud computing resource allocation and management such that required service levels are met. Service Level Agreement (SLA) planning and fulfillment 635 provide pre-arrangement for, and procurement of, cloud computing resources for which a future requirement is anticipated in accordance with an SLA.

Workloads layer 640 provides examples of functionality for which the cloud computing environment can be utilized. Examples of workloads and functions that can be provided from this layer include: mapping and navigation 641; software development and lifecycle management 642; virtual classroom education delivery 643; data analytics processing 644; transaction processing 645; and key biomarker selection and phenotype prediction 646.

The present disclosure may be a system, a method, and/or a computer program product. The computer program product may include a computer readable storage medium (or media) having computer readable program instructions thereon for causing a processor to carry out aspects of the present disclosure.

The computer readable storage medium is a tangible device that can retain and store instructions for use by an instruction execution device. Examples of computer readable storage media can include an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a component, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present disclosure has been described in terms of specific embodiments, it is anticipated that alterations and modification thereof will become apparent to the skilled in the art. Therefore, it is intended that the following claims be interpreted as covering all such alterations and modifications as fall within the true spirit and scope of the present disclosure.

What is claimed is:

1. A system, comprising:
at least one processing component;
at least one memory component;
training data, comprising a set of biomarkers associated with a known phenotype;
a training module, comprising:
a biomarker selector configured to:
receive the set of biomarkers;
generate at least one ranking for each biomarker in the set of biomarkers based on a feature selection method;
select a set of potential key biomarkers from the set of biomarkers based on the at least one rank; and
select a set of key biomarkers from the set of potential key biomarkers; and
a model generator configured to build a model for the known phenotype based on the set of key biomarkers.

2. The system of claim 1, further comprising a testing module configured to:
receive a microbiota sample;
identify, via targeted testing, the set of key biomarkers in the microbiota sample; and
predict, based on the identification, a phenotype associated with the microbiota sample.

3. The system of claim 1, wherein the model generator is further configured to:
apply the model to a subset of the training data;
predict a phenotype associated with the subset of the training data;
evaluate performance of the model based on the predicted phenotype; and
determine that the performance is below a threshold performance value.

4. The system of claim 3, wherein the biomarker selector is further configured to select additional key biomarkers in response to the determination that the performance is below the threshold performance value.

5. The system of claim 1, wherein the set of key biomarkers is selected based on graph-based pruning techniques.

6. The system of claim 1, wherein the at least one ranking comprises a correlation value.

7. The system of claim 1, wherein the biomarker selector is further configured to:
group the set of potential key biomarkers into clusters;
select a potential key biomarker from at least one of the clusters; and
add the selected potential key biomarker to the set of key biomarkers.

8. A method, comprising:
receiving a set of biomarkers associated with a known phenotype;
generating at least one ranking for each biomarker in the set of biomarkers based on a feature selection method;
selecting a set of potential key biomarkers from the set of biomarkers based on the at least one rank;
selecting a set of key biomarkers from the set of potential key biomarkers; and
building a model for phenotype prediction based on the set of key biomarkers.

9. The method of claim 8, further comprising:
receiving a microbiota sample;
identifying, via targeted testing, the set of key biomarkers in the microbiota sample; and
predicting, based on the identification, a phenotype associated with the microbiota sample.

10. The method of claim 8, further comprising:
applying the model to a subset of the training data;
predicting a phenotype associated with the subset of the training data;
evaluating performance of the model based on the testing; and
determining that the performance is below a threshold performance value.

11. The method of claim 10, further comprising selecting additional key biomarkers in response to the determining that the performance is below the threshold performance value.

12. The method of claim 8, wherein the set of key biomarkers is selected based on graph-based pruning techniques.

13. The method of claim 8, wherein the at least one ranking comprises a correlation value.

14. The method of claim 8, further comprising:
grouping the set of potential key biomarkers into clusters;
selecting a potential key biomarker from at least one of the clusters; and
adding the selected potential key biomarker to the set of key biomarkers.

15. A computer program product, the computer program product comprising a computer readable storage medium having program instructions embodied therewith, the program instructions executable by a processor to cause a device to perform a method, the method comprising:
receiving a set of biomarkers associate with a known phenotype;
generating at least one ranking for each biomarker in the set of biomarkers based on a feature selection method;
selecting a set of potential key biomarkers from the set of biomarkers based on the at least one rank;
selecting a set of key biomarkers from the set of potential key biomarkers; and
building a model for the known phenotype based on the set of key biomarkers.

16. The computer program product of claim 15, further comprising:
receiving a microbiota sample;
identifying, via targeted testing, the set of key biomarkers in the microbiota sample; and
predicting, based on the identification, a phenotype associated with the microbiota sample.

17. The computer program product of claim 15, further comprising:
applying the model to a subset of the training data;
predicting a phenotype associated with the subset of the training data;
evaluating performance of the model based on the testing; and
determining that the performance is below a threshold performance value.

18. The computer program product of claim 17, further comprising selecting additional key biomarkers in response to the determining that the performance is below the threshold performance value.

19. The computer program product of claim 15, wherein the set of key biomarkers is selected based on graph-based pruning techniques.

20. The computer program product of claim 15, further comprising:
grouping the set of potential key biomarkers into clusters;
selecting a potential key biomarker from at least one of the clusters; and
adding the selected potential key biomarker to the set of key biomarkers.
